(19) United States
(12) Patent Application Publication
(10) Pub. No.: US 2024/0415702 A1
(43) Pub. Date: Dec. 19, 2024

(54) DRESSING OF TONGUE AND LIP TIES AFTER SURGICAL RELEASE AND CREATION OF ADDITIONAL ATTACHED (KERATINIZED) GINGIVAL TISSUE AROUND TEETH

(71) Applicant: PAGA, LLC, Pepper Pike, OH (US)

(72) Inventor: Vladimir PASTOUK, Mayfield Village, OH (US)

(73) Assignee: PAGA, LLC, Pepper Pike, OH (US)

(21) Appl. No.: 18/740,668

(22) Filed: Jun. 12, 2024

Related U.S. Application Data
(60) Provisional application No. 63/472,746, filed on Jun. 13, 2023.

(51) Int. Cl. A61F 13/0246; A61F 13/00; A61F 13/12

(52) U.S. Cl. CPC A61F 13/0253; A61F 13/122; A61F 2013/00672; A61F 2013/00714; A61F 2013/00719

(57) ABSTRACT

Soft tissue in the mouth (or other body cavity) is surgically released by an incision to free up the tension of the tissue. To prevent premature reattachment of the tissue after separation, a wound dressing is applied to the surgical site followed by application of a substance, malleable during application, that provides a dressing fixture over the wound dressing in an area of the surgical site that is to remain separated. The substance adheres to the area of application and at least partially hardens within a short period of time, then remains in place while the surgical site heals. The dressing fixture may be cyanoacrylate.

### CROSS-REFERENCE TO RELATED APPLICATIONS

This application claims priority to U.S. Provisional Patent Application No. 63/472,746, filed on Jun. 13, 2023 which is incorporated by reference herein in its entirety.

### BACKGROUND

There are elective surgical procedures for releasing tongue and lip muscle and fascia ties and other elective surgical procedures to increase the amount of attached (keratinized) gums around teeth, all of which are independent and may be done at different times to different people. There has been a report by Kazzi and Silverberg of using 2-octyl cyanoacrylate (Dermabond®) to treat tongue lacerations; however, this treatment of tongue lacerations uses Dermabond® in a similar manner to the use of GluStitch® on skin, i.e., to close wounds by “stitching” sides of the wound together which is not the effect desired in the release of tongue ties, lip ties or increasing of attached (keratinized) gums procedure.

### SUMMARY

It is an aspect of the embodiments discussed herein to keep an area of soft tissue (“free gingiva”) open during healing by application of an adhesive over the area of tissue.

Another aspect of the embodiments discussed herein is to apply a wound dressing on the area of soft tissue opened by a scalpel or laser or any other tissue separation technique, prior to application of the adhesive over the area of tissue.

In an embodiment, the wound dressing includes a sodium ascorbate powder.

In an embodiment, the adhesive includes a cyanoacrylate.

### BRIEF DESCRIPTION OF DRAWINGS

These and other aspects and advantages will become more apparent and more readily appreciated from the following description of the exemplary embodiments, taken in conjunction with the accompanying drawings of which:

FIG. 1 illustrates a lip tie;

FIG. 2 illustrates a tongue tie;

FIGS. 3A-3C illustrate a tongue tie that has been opened (FIG. 3A) by a scalpel incision or a laser assisted incision to free up the tension of the tissue, then application of a wound dressing (FIG. 3B), followed by application of a cyanoacrylate, such as PeriAcryl® from GluStitch® (FIG. 3C);

FIG. 4 illustrates a lip tie that has been opened by a scalpel incision or a laser assisted incision to free up the tension of the tissue with application of a cyanoacrylate over a wound dressing; and

FIG. 5 illustrates application of cyanoacrylate to a surgical site on the lower gums after application of a wound dressing.

### DETAILED DESCRIPTION OF EMBODIMENT(S)

The articles “a”, “an” and “the” are intended to include both singular and plural forms of the nouns following that article. The term “and/or” includes a plurality of combinations of relevant items or any one item among a plurality of relevant items. The term “at least [number]” preceding a list of items denotes any combination of the items in the list that satisfies the expression. In the case of “at least one” the expression includes any one item among a plurality of the listed item(s), as well as multiple items. Expressions such as “at least one of,” when preceding a list of elements, modify the entire list of elements and do not modify the individual elements of the list. A phrase of the form “at least one of A, B and C” covers any of the following: A; B; C; A and B; A and C; B and C; and all of A, B and C.

The terms “comprise(ing),” “include(ing),” and “have(ing)” when used in this specification, specify the presence of stated features, functions, processes/operations, elements, and/or components, but do not preclude the presence or addition of one or more other features, functions, processes/operations, elements, components, and/or groups thereof.

Reference will now be made in detail to embodiments, examples of which are illustrated in the accompanying drawings. In this regard, the embodiments may have different forms and should not be construed as being limited to the descriptions set forth herein. In order to further clearly describe features of the embodiments, descriptions of other features that are well known to one of ordinary skill in the art are omitted here.

Elective surgical procedures that release tongue and lip muscle and fascia ties by increasing the surface area and similar procedures can increase strong supportive (keratinized) gingiva around teeth or dental implants. Thus, these procedures are significantly different than the more common situation in which stitches or adhesive(s) are used to close a wound, whether the wound was created by surgery, accident or by an intentional attack. For example, an adhesive substance can be used in the mouth permitting tissue to heal only to the extent desired by applying the adhesive over a wound dressing. The procedure is also applicable to other situations in which it is desirable to prevent premature reattachment of tissue after its separation, particularly in the mouth or other cavities or parts of the body.

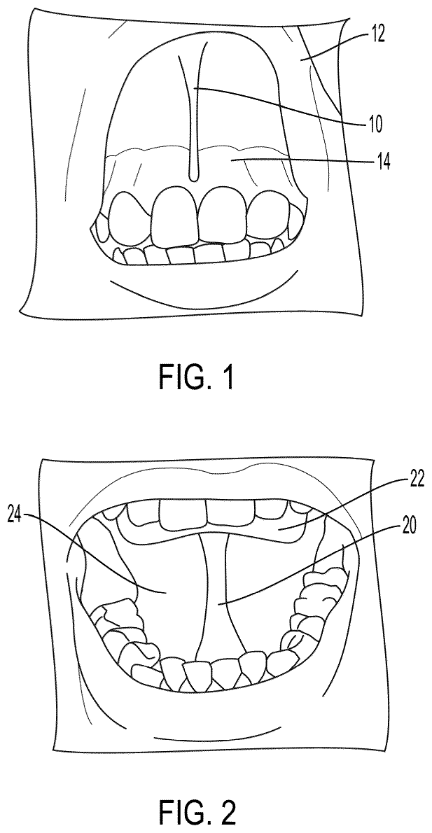

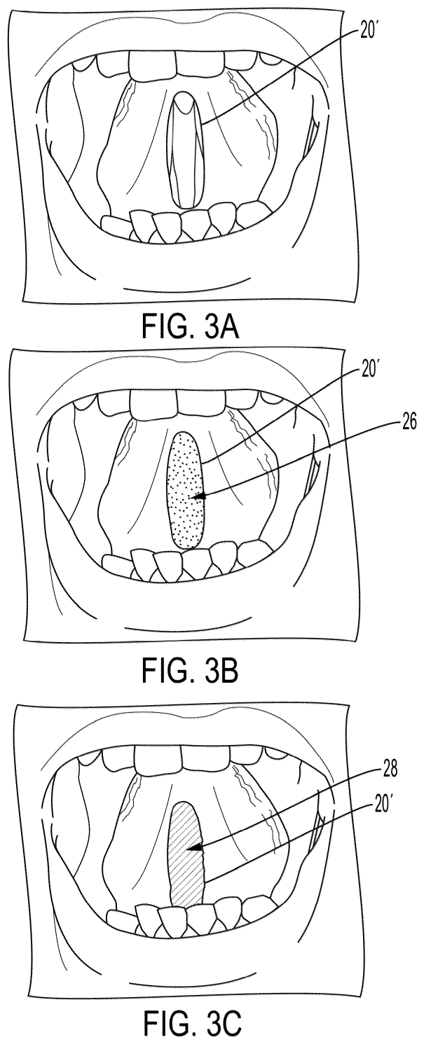

Illustrated in FIG. 1 is a lip tie 10 between the upper lip 12 and gums 14. Illustrated in FIG. 2 is a tongue tie 20 between the tongue 22 and the bottom of the mouth 24. Both ties 10 and 20 are formed of soft tissue. To release such ties, an incision is made by a scalpel or laser along the center edge. As illustrated in FIG. 3A, this results in a surgical site 20′ in the case of a tongue tie.

According to an embodiment illustrated in FIG. 3B, a wound dressing 26 which may include sodium ascorbate powder is applied over the surgical site 20′. The structure of sodium ascorbate is

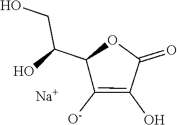 [SMILES: O=C1O[C@H]([C@@H](O)CO)C([O-])=C1O ; [Na+]]

Next, as illustrated in FIG. 3C, a dressing fixture 28 that is malleable during application, is applied over the wound dressing 26 (FIG. 3B) at the surgical site 20′ to ensure the stability of the wound dressing. To perform its function properly, the dressing fixture 28 is a substance that will maintain its shape and stay in place while the surgical site 20′ heals. To accomplish this function, it is desirable for the substance to substantially harden, although it may retain some flexibility, and be non-adhesive to other tissue in the mouth after a period of time. In an embodiment, the period of time is five minutes or less.

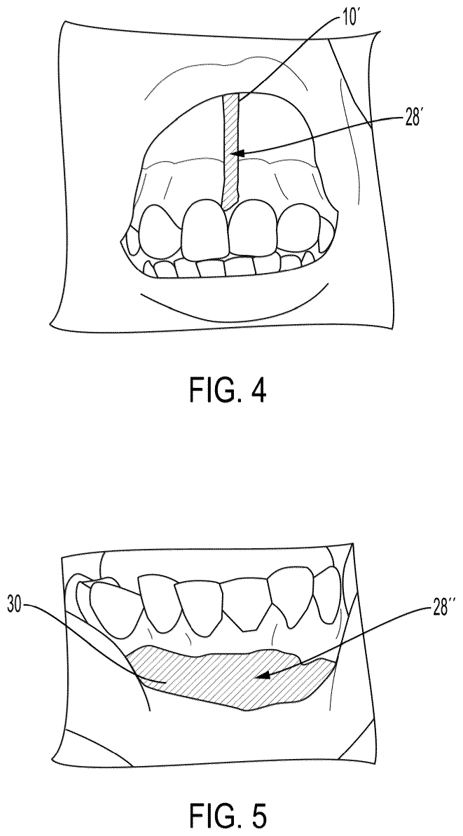

Similarly, a dressing fixture 28′ can be applied over a wound dressing at a surgical site 10′ for a lip tie, as illustrated in FIG. 4. In addition, a dressing fixture can be applied over a wound dressing elsewhere in the mouth in other surgical procedures besides loosening tongue and lip ties. An example is illustrated in FIG. 5 for application of a dressing fixture 28″ over a wound dressing after an elective surgical procedure to increase the amount of attached (keratinized) gums around teeth at a surgical site 30.

According to an embodiment, the dressing fixture is an adhesive that is suitable for use in the mouth. In the U.S.A., the composition of the dressing fixture should be approved for oral use by the Food and Drug Administration (FDA). According to an embodiment, the adhesive includes an cyanoacrylate, such as PeriAcryl® from GluStitch®, which is available in 90/10 butyl/octyl, 80/20 butyl/octyl, 70/30 butyl/octyl and 60/40 butyl/octyl formulations and is approved by the FDA for use as an adjunct to temporarily assist in securing periodontal dressings. The dressing fixture used in the embodiments illustrated in FIGS. 3C, 4 and 5 may be 90/10 butyl/octyl PeriAcryl®.

The many features and advantages of the embodiments are apparent from the detailed specification and, thus, it is intended by the appended claims to cover all such features and advantages of the embodiments that fall within the true spirit and scope thereof. Further, since numerous modifications and changes will readily occur to those skilled in the art, it is not desired to limit the inventive embodiments to the exact construction and operation illustrated and described, and accordingly all suitable modifications and equivalents may be resorted to, falling within the scope thereof.

What is claimed is:

1. A surgical procedure performed on separated tissue, comprising:
applying a wound dressing to the separated tissue; and
applying a substance, malleable during application, over the wound dressing on an area of the separated tissue desired to remain separated during healing, the substance hardening after application.

2. The surgical procedure as in claim 1, wherein the substance providing a dressing fixture.

3. The surgical procedure as in claim 2, wherein the dressing fixture is a cyanoacrylate adhesive.

4. The surgical procedure as in claim 3, wherein the cyanoacrylate becomes substantially non-adhesive within five minutes.
